(19) United States
(12) Patent Application Publication
(10) Pub. No.: US 2018/0368477 A1
(43) Pub. Date: Dec. 27, 2018

(54) INHALER DEVICE AND METHOD OF OPERATING SAME

(71) Applicant: JT International S.A., Geneva (CH)

(72) Inventors: Andrew John Ede, Cambridge (GB); Russell Graham Farn, Cambridge (GB); Rebecca Ann Wilkins, Saffron Walden (GB); Alexander Laurence Anderton, Preston (GB)

(73) Assignee: JT International S.A., Geneva (CH)

(21) Appl. No.: 16/062,002

(22) PCT Filed: Dec. 13, 2016
(86) PCT No.: PCT/EP2016/080730

§ 371 Date: Jun. 13, 2018

(30) Foreign Application Priority Data
Dec. 18, 2015 (EP) 15201253.0

(51) Int. Cl. A24F 47/00; A61M 15/00; A61M 15/06; A61M 11/04

(52) U.S. Cl. CPC A24F 47/008; A61M 15/0023; A61M 2205/8206; A61M 11/042; A61M 2205/276; A61M 15/06

(57) ABSTRACT

An inhaler device, such as a personal vaporizer or an electronic smoking article, for generating vapour (V) for inhalation includes a body portion for generating the vapour for inhalation; and a mouthpiece provided on the body portion for insertion into the mouth of a user and including at least one channel for conveying the vapour to the user. The mouthpiece is configured for movement relative to the body portion, especially translational movement and/or rotational movement, between a first position and a second position in order to switch the device between a deactivated state and an activated state, respectively. A corresponding method of operating an inhaler device, such as an electronic cigarette is also provided.

### FIELD OF THE INVENTION

The present invention relates to an inhaler device, such as an electronic cigarette (e-cigarette), a personal vaporizer or an electronic vapour delivery system. More particularly, the invention relates to a heating system for such an inhaler device and a method of heating for generating an aerosol or a vapour from a substance to be heated in such a device.

### BACKGROUND OF THE INVENTION

Inhaler devices of the above types, namely e-cigarettes and personal vaporizers and electronic vapour delivery systems, are proposed as an alternative to traditional smoking articles, such as cigarettes, cigarillos, cigars and the like. Typically, these inhaler devices are designed to heat a liquid solution or a gel to produce or generate an aerosol and/or a vapour to be inhaled by a user. This liquid or gel is usually a solution of propylene glycol (PG) and/or vegetable glycerin (VG), and typically contains a flavorant or one or more concentrated flavours.

Despite the increasing demand for these inhaler devices and the growing market, efforts are still required to develop the performance of these devices, with a view to offering more efficient and improved products. For example, these efforts are directed to improved aerosol and/or vapour generation and delivery, especially to more efficient energy use in aerosol and/or vapour generation to improve energy consumption, e.g. to enhance battery life of the device, ergonomics and ease of operation, as well as child-resistance aspects of the device.

### SUMMARY OF THE INVENTION

In view of the above, an object of the invention is to provide a new and improved inhaler device, especially an electronic cigarette, and a new and improved method of operating such a device, and especially providing the inhaler device with child-resistance features.

In accordance with the invention, an inhaler device having the features recited in claim 1 and a method of operating an inhaler device as recited in claim 11 are provided. Various advantageous and/or preferred features of the present invention are recited in the dependent claims.

According to one aspect, therefore, the invention provides an inhaler device, such as a personal vaporizer or an electronic smoking article, for generating vapour for inhalation, the device comprising: a body portion; and a mouthpiece provided on the body portion for insertion into the mouth of a user and including at least one channel for conveying the vapour to the user. The mouthpiece is configured for movement relative to the body portion, or at least part of the body portion, between a first position and a second position to switch the device between a deactivated state and an activated state, respectively. Thus, in the activated state, the device is effectively switched “on” and is able to generate the vapour for inhalation. In the deactivated state, however, the device is effectively switched “off” and unable to generate the vapour for inhalation.

In this way, the present invention provides an inhaler device having a mechanism for switching the device between a deactivated state and an activated state via a relative movement of the mouthpiece and the body portion. In this regard, it will be noted that, because the relevant movement is specifically recited as a “relative” movement, either the mouthpiece may move with respect to the body portion, or the body portion may move with respect to the mouthpiece to generate or provide the required movement. These parts—i.e. mouthpiece and body portion—of the inhaler device may be moved relative to one another by hand. Alternatively, they may be moved relative to one another when the mouthpiece is in the user's mouth, thereby combining e.g. mouth and hand actuated or effected movement. The fact that these parts of the device need to be moved relative to one another, optionally combined with a threshold level of force required to effect the relative movement, may furthermore serve as a child-resistant feature inhibiting inadvertent activation, or possibly even an intended activation, of the device by a young child.

In a preferred embodiment, the body portion is generally elongate and defines a longitudinal axis for the device. In this context, the mouthpiece may be configured for movement relative to the body portion at least partially in the axial direction between the first and the second positions. In other words, the mouthpiece may have to be displaced in the axial direction relative to the body portion in order to move the mouthpiece to the activated state in the second position.

In a preferred embodiment, the body portion is generally elongate and defines a longitudinal axis for the device. In this context, the mouthpiece may be configured for movement relative to the body portion at least partially in rotation about the axis between the first and the second positions. That is, the mouthpiece may have to be rotated about the longitudinal axis relative to the body portion in order to move the mouthpiece to the activated state in the second position. It will be appreciated that the mouthpiece may be configured for movement relative to the body portion in a combination of both axial displacement and rotation about the axis between the first and the second positions. Relative movements of the mouthpiece and the body portion, especially translational and/or rotational relative movements, may be contemplated in other directions also.

In a preferred embodiment, the mouthpiece comprises a sleeve member which surrounds an end region of the body portion, especially an end region of a casing of the body portion. The sleeve member is configured to be displaced axially and rotated about the axis for movement relative to the body portion between the first and second positions. In this context, the sleeve member may include a slot which defines a path for the relative movement of the mouthpiece between the first and second positions. Preferably, the body portion includes a follower element, such as a pin, which may be registered in the slot. In this way, opposite ends of the slot may therefore define the respective first and second positions of the mouthpiece relative to the body portion.

In a preferred embodiment, the mouthpiece is resiliently biased to the first position via bias means, such as a spring (e.g. a compression spring or a torsion spring), to maintain the device in the deactivated state against a predetermined biasing force. In this regard, the predetermined biasing force is preferably selected to exceed a typical or expected manual strength of a young child in order to render switching of the device to an activated state child-resistant.

In a preferred embodiment, the body portion of the inhaler device comprises one or more of: a reservoir or receptacle for holding or storing a substance, such as a liquid, to be vaporized; an aerosol generating means for vaporizing the substance; a power supply for supplying power, and especially electrical power, to the aerosol generating means; and/or a control unit for controlling operation of the aerosol generating means. Thus, in the first position, an electrical connection between the power supply and aerosol generating means may be interrupted to switch the device to the deactivated state. On the other hand, in the second position, the electrical connection between the power supply and the aerosol generating means may be completed to switch the device to the activated state. Preferably, at least the power supply (e.g. battery unit) for supplying electrical power is provided in the body portion of the inhaler device. One or more of a reservoir for holding or storing the liquid to be vaporized; an aerosol generating means for vaporizing the liquid; and/or a control unit for controlling operation of the aerosol generating means, may optionally be provided in the mouthpiece of the inhaler device.

Thus, in a preferred embodiment, the elongate body portion may include aerosol generating means for generating the vapour for inhalation. On the other hand, in an alternative embodiment, the mouthpiece may include the aerosol generating means for generating the vapour for inhalation.

According to another aspect, the present invention provides a method of operating an inhaler device, such as an electronic cigarette, comprising a body portion, and a mouthpiece provided on the body portion for insertion into a mouth of a user and having at least one channel for conveying vapour to the user. The method comprises: moving the mouthpiece relative to the body portion, or vice versa, between a first position and a second position to switch the device between a deactivated state and an activated state, respectively. As noted above, the device is unable to generate vapour for inhalation in the deactivated state, whereas in the activated state, the device is able to generate vapour for inhalation.

In a preferred embodiment, the step of relatively moving the mouthpiece to the first position interrupts an electrical connection between a power supply and an aerosol generating means in the body portion to switch the device to the deactivated state.

Furthermore, the step of relatively moving the mouthpiece to the second position completes the electrical connection between the power supply and the aerosol generating means to switch the device to the activated state.

As noted above, in a preferred embodiment the body portion is generally elongate and defines a longitudinal axis for the device, whereby the step of moving the mouthpiece relative to the body portion between the first and the second positions comprises a movement at least partially in the axial direction. Alternatively, or in addition, the step of moving the mouthpiece relative to the body portion between the first and the second positions may comprise a movement at least partially in rotation about the axis. In other words, moving the mouthpiece relative to the body portion between the first and the second positions may comprise a combination of a translational and a rotational movement; for example, the relative movement may be along and/or around the longitudinal axis of the elongate body portion.

### BRIEF DESCRIPTION OF THE DRAWINGS

For a more complete understanding of the invention and the advantages thereof, exemplary embodiments of the invention are explained in more detail in the following description with reference to the accompanying drawing figures, in which like reference characters designate like parts and in which:

FIG. 1a is a schematic perspective view of an inhaler device according to one embodiment being operated by a user;

FIG. 1b is a schematic perspective view of an inhaler device according to another embodiment being operated by a user;

FIG. 2 is a schematic perspective view of part of an inhaler device according to an embodiment in a first position in a deactivated state;

FIG. 3 is a longitudinal cross-sectional view of the part of the inhaler device of FIG. 2 in the first position in the deactivated state;

FIG. 4 is a schematic perspective view of the part of the inhaler device of FIG. 2 in a second position in an activated state;

FIG. 5 is a longitudinal cross-sectional view of the part of the inhaler device of FIG. 4 in the second position in the activated state; and

FIG. 6 is a flow diagram which schematically represents a method according to an embodiment of the invention.

The accompanying drawings are included to provide a further understanding of the present invention and are incorporated in and constitute a part of this specification. The drawings illustrate particular embodiments of the invention and together with the description serve to explain the principles of the invention. Other embodiments of the invention and many of the attendant advantages of the invention will be readily appreciated as they become better understood with reference to the following detailed description.

It will be appreciated that common and/or well understood elements that may be useful or necessary in a commercially feasible embodiment are not necessarily depicted in order to facilitate a more abstracted view of the embodiments. The elements of the drawings are not necessarily illustrated to scale relative to each other. It will further be appreciated that certain actions and/or steps in an embodiment of a method may be described or depicted in a particular order of occurrences while those skilled in the art will understand that such specificity with respect to sequence is not actually required. It will also be understood that the terms and expressions used in the present specification have the ordinary meaning as is accorded to such terms and expressions with respect to their corresponding respective areas of inquiry and study, except where specific meanings have otherwise been set forth herein.

### DETAILED DESCRIPTION OF EMBODIMENTS

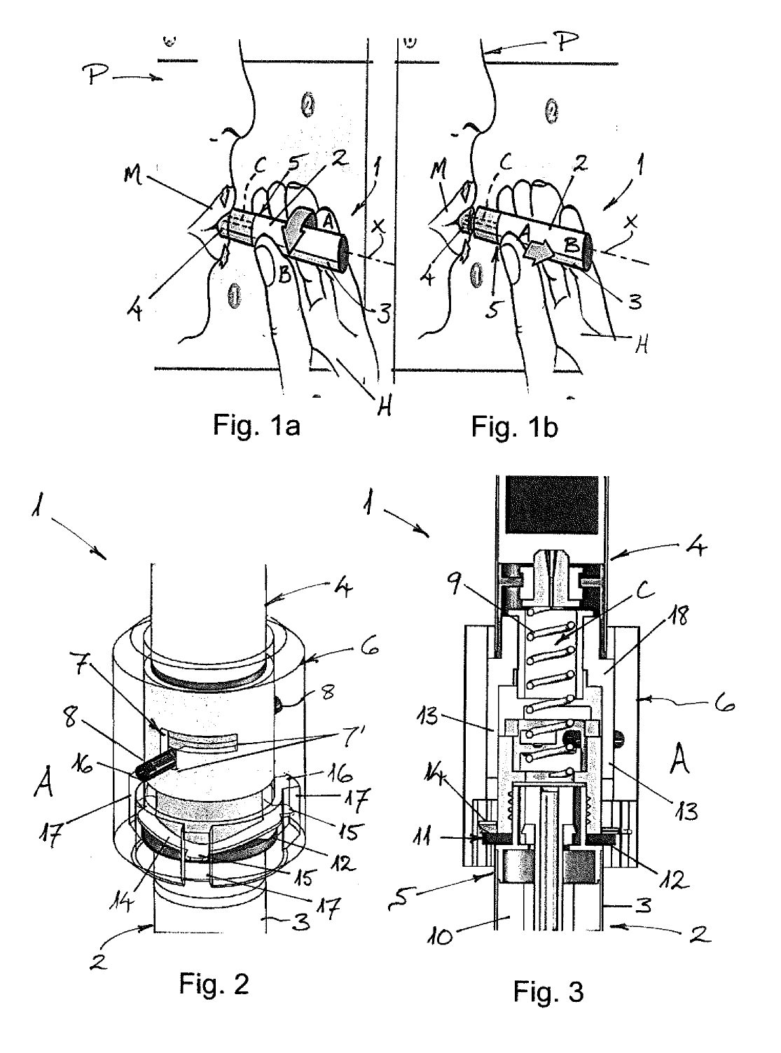

With reference to FIG. 1a of the drawings, one embodiment of an inhaler device 1 is illustrated schematically in use or in operation by a user P. The inhaler device 1 is in the form of an electronic cigarette or e-cigarette which comprises an elongate body portion 2 having a longitudinal or central axis X. The body portion 2 has a generally cylindrical casing 3 which encloses or houses one or more of a reservoir (not shown) for holding or storing a liquid to be vaporized, an aerosol generating means (not shown) for vaporizing the liquid to generate a vapour V for inhalation, a power supply such as a battery unit (not shown) for supplying electrical power to the aerosol generating means, and a control unit for controlling operation of the aerosol generating means. A mouthpiece 4 is provided at one end region 5 of the body portion 2 for insertion into the mouth M of the user P and includes at least one channel C for conveying the vapour to the user as the user inhales or draws on the mouthpiece 4. It will be appreciated, however, that in other embodiments, the aerosol generating means (not shown) for vaporizing the liquid to generate the vapour V for inhalation and/or the control unit (not shown) for controlling operation of the aerosol generating means may be incorporated in the mouthpiece 4.

In the embodiment of FIG. 1a, the mouthpiece 4 of the device 1 is configured for rotational movement relative to the body portion 2 about the longitudinal or central axis X between a first position A and a second position B to switch the device 1 between a deactivated state and an activated state, respectively. In this regard, FIG. 1a illustrates that, in operation, a user P can hold the mouthpiece 4 in his/her mouth M in a first action (labelled “1” in circle) and then manually (i.e. by hand H) rotate the body portion 2 relative to the mouthpiece 4 in a second action (labelled “2” in circle) to switch the device 1 via the first and second positions between the deactivated and activated states.

Referring now to FIG. 1b of the drawings, another embodiment of an inhaler device 1 is shown schematically in use or in operation by a user P. In this embodiment, the general form or configuration of the inhaler device 1 is substantially the same as in FIG. 1a, with like reference signs designating like parts. In this embodiment, however, the mouthpiece 4 of the device 1 is configured for axial or translational movement relative to the body portion 2 along the longitudinal or central axis X between a first position A and a second position B to switch the device 1 between a deactivated state and an activated state, respectively. Accordingly, in operation FIG. 1b shows that a user P can hold the mouthpiece 4 in his/her mouth M in a first action (labelled “1” in circle) and then manually (i.e. by hand H) draw or pull the body portion 2 relative to the mouthpiece 4 in the axial direction in a second action (labelled “2” in circle) to move the device 1 from the first position A to the second position B to switch from the deactivated state to the activated state. Pushing the body portion 2 in the axial direction back towards the mouthpiece 4 can then return the mouthpiece 4 to the first position A to switch the device 1 back to the deactivated state.

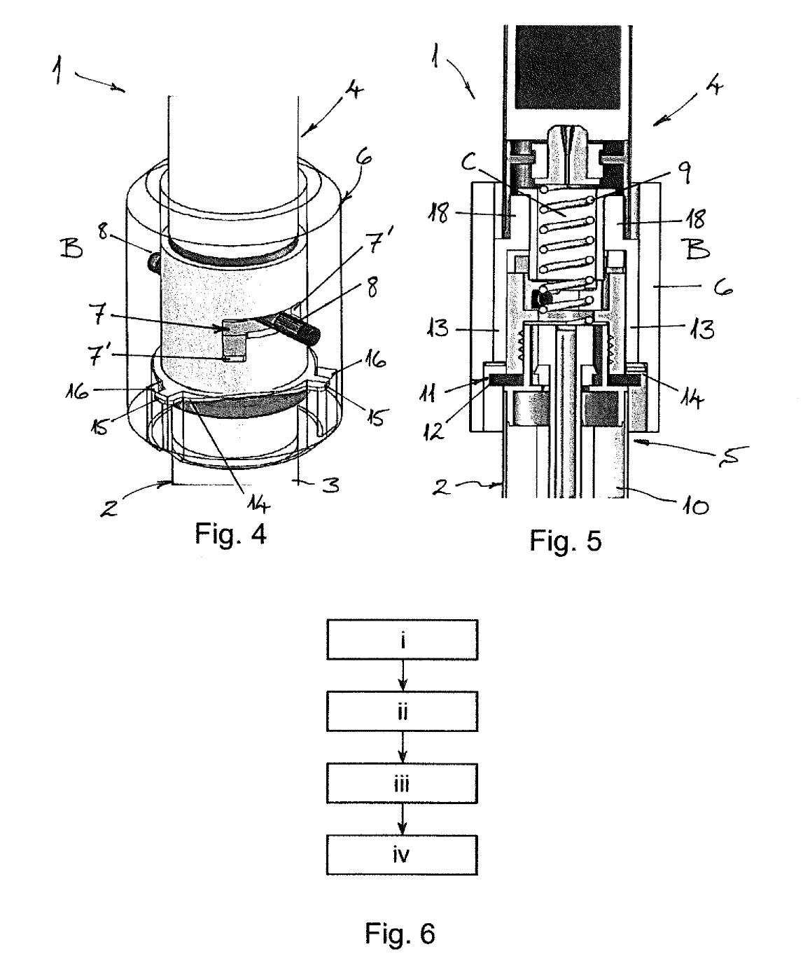

With reference now to FIGS. 2 to 5 of the drawings, part of an inhaler device or an e-cigarette 1 according to a further embodiment is illustrated showing details of the mechanism for effecting the relative movement between the mouthpiece 4 and the body portion 2 between the first and second positions A, B. In this embodiment, the relative movement between the mouthpiece 4 and the body portion 2 of the device 1 combines both displacement in the axial direction and rotation about the longitudinal axis X; i.e. a push or pull combined with a twist. In this regard, drawing FIGS. 2 and 3 show the e-cigarette device 1 in the first position A in the deactivated state, whereas FIGS. 4 and 5 show the device 1 in the second position B and in the activated state.

Referring to FIGS. 2 and 3, it will be seen that the mouthpiece 4 includes a sleeve member 6 which surrounds the end region 5 of the body portion 2, especially the end region of the casing 3. The sleeve member 6 is configured to be both axially displaced and rotated about the axis X for movement relative to the body portion 2 between the first and second positions A, B. In this context, the sleeve member 6 includes a slot 7 which effectively defines a path for the relative movement of the mouthpiece 4 between the first and second positions A, B. Furthermore, the body portion 2 includes a follower element 8, such as a pin, which registers in the slot 7 such that opposite ends 7′ of the slot 7 then delimit or define the respective first and second positions A, B of the mouthpiece 4 relative to the body portion 2. Within the sleeve member 6, a compression spring 9 is provided to resiliently bias the mouth-piece 4 to the first position A. In this way, the spring 9 acts to maintain the device 1 in the deactivated state against a predetermined spring biasing force, which may be selected to exceed a typical or expected manual strength of a child in order to render switching of the device to an activated state child-resistant.

In this embodiment, the casing 3 of the body portion 2 encloses or houses a power supply 10, such as a battery unit, which connects electrically with a control unit 11 provided on a printed circuit board 12 arranged at the end region 5 of the casing 3 and covered by the sleeve member 6. When the mouthpiece 4 with the sleeve member 6 is moved axially against the bias of the spring 9 and rotationally via the slot 7 and pin 8 to the second position B shown in FIGS. 4 and 5, the coil spring 9 is compressed and an electrical connector element 13, here in the form of a sheath surrounding the central channel C of the mouthpiece 4 within the sleeve member 6, moves into contact with a wave-type spring washer 14. The flexible wave-type spring washer 14 is thereby pressed into electrical contact with the printed circuit board 12 of the control unit 11, as lobe- or tongue-like projections 15 on the spring washer 14 engage with upper ends 16 of grooves 17 on an inner side of the sleeve member 6. In this way, an aerosol generating means 18 for vaporizing liquid stored in a reservoir (not shown) is electrically connected and activated in the second position B via its connection with the sheath 13, and thus to the battery unit 10, to generate vapour for inhalation. The pin 8 residing in the slot 7, as shown in FIG. 4, then holds the mouthpiece 4 in the second position B.

Finally, referring to FIG. 6 of the drawings, a flow diagram is shown that illustrates schematically the steps in a method of operating an inhaler device 1, such as an electronic cigarette, according to an embodiment of the invention described above with respect to FIGS. 1 to 5. In this regard, the first box i of FIG. 6 represents the step of providing the inhaler device 1 comprising a body portion 2 for generating vapour for inhalation, and a mouthpiece 4 on the body portion 2 for insertion into the mouth of a user and having at least one channel C for conveying the vapour to the user. The second box ii then represents the step of moving the mouthpiece 4 in a translational and/or rotational manner relative to the body portion 2 from the first position A to a second position B to switch the device 1 from a deactivated state to an activated state. The third box iii represents the step of generating and inhaling vapour with the device in the activated state. The final box iv in FIG. 6 of the drawings represents the optional step of again moving the mouthpiece 4 in a translational and/or rotational manner relative to the body portion 2 back from the second position B to the first position A to switch the device 1 from the activated state back to the deactivated state.

Although specific embodiments of the invention are illustrated and described herein, it will be appreciated by those of ordinary skill in the art that a variety of alternate and/or equivalent implementations exist. It should be appreciated that the exemplary embodiment or exemplary embodiments are examples only and are not intended to limit the scope, applicability, or configuration in any way. Rather, the foregoing summary and detailed description will provide those skilled in the art with a convenient road map for implementing at least one exemplary embodiment, it being understood that various changes may be made in the function and arrangement of elements described in an exemplary embodiment without departing from the scope as set forth in the appended claims and their legal equivalents. Generally, this application is intended to cover any adaptations or variations of the specific embodiments discussed herein.

It will also be appreciated that in this document the terms “comprise”, “comprising”, “include”, “including”, “contain”, “containing”, “have”, “having”, and any variations thereof, are intended to be understood in an inclusive (i.e. non-exclusive) sense, such that the process, method, device, apparatus or system described herein is not limited to those features or parts or elements or steps recited but may include other elements, features, parts or steps not expressly listed or inherent to such process, method, article, or apparatus. Furthermore, the terms “a” and “an” used herein are intended to be understood as meaning one or more unless explicitly stated otherwise. Moreover, the terms “first”, “second”, “third”, etc. are used merely as labels, and are not intended to impose numerical requirements on or to establish a certain ranking of importance of their objects.

### LIST OF DRAWING SIGNS

1 an inhaler device or e-cigarette

2 body portion

3 casing

4 mouthpiece

5 end region of body portion

6 sleeve member

7 slot

7′ end of slot

8 follower element or pin

9 compression spring

10 power supply or battery unit

11 control unit

12 printed circuit board

13 electrical connector element or sheath

14 spring washer

15 lobe- or tongue-like projection

16 upper end of groove

17 groove

18 aerosol generating means

X longitudinal axis or central axis

A first position

B second position

C channel

P user

M mouth

H hand

V vapour

1. An inhaler device, such as a personal vaporizer or an electronic smoking article, for generating vapour for inhalation, the device comprising:
a body portion; and
a mouthpiece provided on the body portion for insertion into the mouth of a user and including at least one channel for conveying vapour generated to the user;
wherein the mouthpiece is configured for movement relative to the body portion, or at least part thereof, between a first position and a second position to switch the device between a deactivated state and an activated state in which the device is able to generate the vapour for inhalation, respectively.

2. An inhaler device according to claim 1, wherein the body portion is generally elongate and defines a longitudinal axis for the device, and wherein the mouthpiece is configured for movement relative to the body portion at least partially in the axial direction between the first and the second positions.

3. An inhaler device according to claim 1, wherein the body portion is generally elongate and defines a longitudinal axis for the device, and wherein the mouthpiece is configured for movement relative to the body portion at least partially in rotation about the axis between the first and the second positions.

4. An inhaler device according to any one of claims 1, wherein the mouthpiece is resiliently biased to the first position via compression spring to maintain the device in the deactivated state against a predetermined biasing force.

5. An inhaler device according to claim 4, wherein the predetermined biasing force is selected to exceed an expected manual strength of a child to render switching of the device to an activated state child resistant.

6. An inhaler device according to claim 4, wherein the mouth-piece comprises a sleeve member which surrounds an end region of the body portion, wherein the sleeve member is configured to be displaced axially and rotated about ta longitudinal axis defined by the body portion for movement relative to the body portion between the first and second positions.

7. An inhaler device according to claim 6, wherein the sleeve member includes a slot defining a path for the movement of the mouthpiece between the first and second positions.

8. An inhaler device according to claim 7, wherein the body portion includes a follower element, registered in the slot, and wherein opposite ends of the slot define the respective first and second positions for the mouthpiece relative to the body portion.

9. An inhaler device according to claim 1, wherein the body portion comprises one or more of:
a reservoir or receptacle for holding or storing a substance to be vaporized;
aerosol generating means for vaporizing the substance;
a power supply for supplying power, especially electrical power, to the aerosol generating means; and
a control unit for controlling operation of the aerosol generating means.

10. An inhaler device according to claim 9, wherein, in the first position, an electrical connection between the power supply and the aerosol generating means is interrupted to switch the device to the deactivated state, and wherein, in the second position, the electrical connection between the power supply and the aerosol generating means is completed to switch the device to the activated state.

11. A method of operating an inhaler device, such as an electronic cigarette, comprising a body portion, and a mouthpiece provided on the body portion for insertion into the mouth of a user and having at least one channel for conveying vapour to the user, the method comprising:
moving the mouthpiece relative to the body portion between a first position and a second position in order to switch the device between a deactivated state and an activated state, respectively.

12. A method according to claim 11, wherein moving the mouthpiece to the first position interrupts an electrical connection between a power supply and an aerosol generating means in the body portion to switch the device to the deactivated state, and wherein moving the mouthpiece to the second position completes the electrical connection between the power supply and the aerosol generating means to switch the device to the activated state.

13. A method according to claim 11, wherein the body portion is generally elongate and defines a longitudinal axis for the device, and wherein the moving of the mouthpiece relative to the body portion between the first and the second positions is at least partially in the axial direction.

14. A method according to claim 11, wherein the body portion is generally elongate and defines a longitudinal axis for the device, and wherein the moving of the mouthpiece relative to the body portion between the first and the second positions is at least partially in rotation about the axis.

15. An inhaler device according to claim 4, wherein the mouth-piece comprises a sleeve member which surrounds an end region of a casing the body portion, wherein the sleeve member is configured to be displaced axially and rotated about a longitudinal axis defined by the body portion for movement relative to the body portion between the first and second positions.

16. An inhaler device according to claim 8, wherein the follower element is a pin.
